(19) United States
(12) Patent Application Publication
(10) Pub. No.: US 2024/0139445 A1
(43) Pub. Date: May 2, 2024

(54) Automated Bag Valve Mask

(71) Applicant: Spir Medical Devices, LLC, Raleigh, NC (US)

(72) Inventors: John Twiddy, Raleigh, NC (US); Taylor Gabaldon, Morrisville, NC (US)

(21) Appl. No.: 18/384,672

(22) Filed: Oct. 27, 2023

Related U.S. Application Data
(60) Provisional application No. 63/420,351, filed on Oct. 28, 2022.

(51) Int. Cl. A61M 16/00

(52) U.S. Cl. CPC A61M 16/0078; A61M 16/0051; A61M 16/024; A61M 2205/3331; A61M 2205/502; A61M 2230/42

(57) ABSTRACT

A patient ventilation system comprising a BVM that is operable in both a manual operating mode and an autonomous operating mode. In the manual operating mode the user operates the BVM manually. Sensors collect data from which performance metrics are calculated and displayed to the user. The displayed metrics (e.g., airway pressure, tidal volume delivered, ventilation rate, and gas concentrations) provide responders with performance feedback allowing them to correct inadequate ventilation. Once the user is satisfied with their performance, they can then enable automated ventilation to provide consistent ventilation to the patient. The controller recognizes the user's technique and with the press of a button, the control system of the DIVA mirrors the performance of the user without requiring separate manual entry of target breathing parameters.

### RELATED APPLICATIONS

This application claims the benefit of U.S. Provisional Application No. 63/420,351, filed Oct. 28, 2022, the entire disclosure of which being hereby incorporated by reference herein.

### TECHNICAL FIELD

The present disclosure relates generally to a bag valve mask (BVM) to deliver positive pressure ventilation to a patient having trouble breathing and, more particularly, to automate control of a BVM.

### BACKGROUND

Artificial ventilation has been a medical practice for a long time, with some of the earliest reports in the mid-18th century. The earliest bag valve mask (BVM) was introduced in 1956 and nicknamed the “Ambu” (Artificial Manual Breathing Unit). A BVM is a medical device used to provide positive pressure ventilation to patients who are unable to breathe unassisted or need an influx of oxygen. The bag valve mask comprises a flexible air chamber or bag attached to a face mask via a shutter valve. When the BVM is properly applied to a patient's face and the bag is squeezed, air is forced through the valve and into the patient's lung. When the bag is released, air with depleted oxygen levels is drawn while also allowing the patient's lungs to deflate to the ambient environment, especially in single user scenarios.

Due to its popular usage in emergency settings, many innovations have been attempted and some have become a part of the BVM, such as antiviral filters and one-way exhalation valves. This has become increasingly prevalent with the arrival and subsequent effects of COVID-19. However, problems, complications, and shortcomings remain in traditional BVM designs that need to be addressed BVM users within emergency medical services (EMS) and clinical emergency medicine (EM) need a way to gauge tidal volume and internal lung pressure delivered in order to prevent patient injury. Additionally, BVM users within EMS and clinical EM need a way to reduce operator fatigue during extended operation.

### SUMMARY

The present disclosure provides a patient ventilation system comprising a BVM that is operable in both a manual operating mode and an autonomous operating mode. In the manual operating mode the user operates the BVM manually. Sensors collect data from which performance metrics are calculated and displayed to the user. The displayed metrics (e.g., airway pressure, tidal volume delivered, ventilation rate, and gas concentrations) provide responders with performance feedback allowing them to correct inadequate ventilation. Once the user is satisfied with their performance, they can then enable automated ventilation to provide consistent ventilation to the patient. The controller recognizes the user's technique and with the press of a button, the control system of the DIVA mirrors the performance of the user.

Automated ventilation fulfills the role of one responder (squeezing the bag at an adequate respiratory rate) allowing a single provider to focus on ensuring leak-proof mask seal around the patient's nose and mouth. This, in combination with sensor feedback to ensure adequate ventilation, minimizes the physical and mental strain on responders and improves patient outcomes.

### BRIEF DESCRIPTION OF THE DRAWINGS

FIG. 1 is a block diagram of a patient ventilation system according to one embodiment.

FIG. 2 is a schematic diagram of a bag valve mask used in the patient ventilation system in an expanded state.

FIG. 3 is a schematic diagram of a bag valve mask used in the patient ventilation system in a collapsed state.

FIG. 4 is a state diagram illustrating operating states or modes.

FIG. 5 schematically illustrates feedback control for the BVM.

FIG. 6A is an exemplary control procedure performed by the controller.

FIG. 6B shows control logic for an exemplary manual control loop.

FIG. 6C shows control logic for an exemplary autonomous control loop.

### DETAILED DESCRIPTION

A common problem encountered by users of traditional BVMs is that traditional BVMs can often exceed the upper threshold of the appropriate volume of air delivery, which could easily put a patient in a critical condition by causing lung injuries or other related conditions. One reason why traditional BVMs fail to deliver an appropriate amount of air is because, although the simplicity of BVMs allows for rapid deployment and use, lack of tidal volume delivery measurements often forces the operator to rely on ambiguous metrics such as chest rise. Such metrics can be quite difficult to rely upon depending on the body mass index of the patient. Thus, one advantage of certain embodiments discussed herein is a BVM design that will protect patients from erroneous use of the BVM that may end up in critical injuries.

In traditional BVMs, mask seal around a patient's face is achieved using two hands gripping around the mask and pulling up the patient's chin. However, in an emergency setting, it is common for only one medical provider to operate a BVM. This can make achieving appropriate mask-seal difficult. Further, the operation of squeezing a BVM over long durations of time can cause operator physical and emotional strain. Thus, another advantage of one or more embodiments discussed herein is a BVM design that facilitates ease of operation, particularly over protracted periods of time.

In general, a BVM is used for 15-45 minutes in one setting until they can switch to another provider or connect to hospital-use ventilation systems. In such a scenario, the provider is not only using the BVM by squeezing the bag every 6 to 8 seconds while keeping a good airway and mask seal, but is also monitoring other vitals and maneuvering calls to hospitals and other tasks, possibly in a moving, noisy, and bumpy ambulance ride. The mental and physical task load of an Emergency Medical Technician (EMT) or Paramedic is greatly challenged, given that they may be the only hope to keep a patient alive. Thus, mask seal leakage, the inability to gauge tidal volume delivery, and user physical and emotional strain became a significant priority in the development of compatible solutions. Embodiments of the present disclosure address at least some of these problems while also mitigating the frequency and/or extent of lung injuries caused by misuse of BVM.

In particular, embodiments of the present disclosure include a BVM comprising a sensor-based patient feedback system to provide operators with performance metrics as well as semi- or fully automatic assistance in operating the BVM. One or more such embodiments advantageously solve the pain points described above by freeing at least one hand from constantly squeezing the bag of a traditional BVM. Consequently, a BVM user can use a freed hand to ensure better mask seal, and/or use other equipment to ensure safe delivery of oxygenated air.

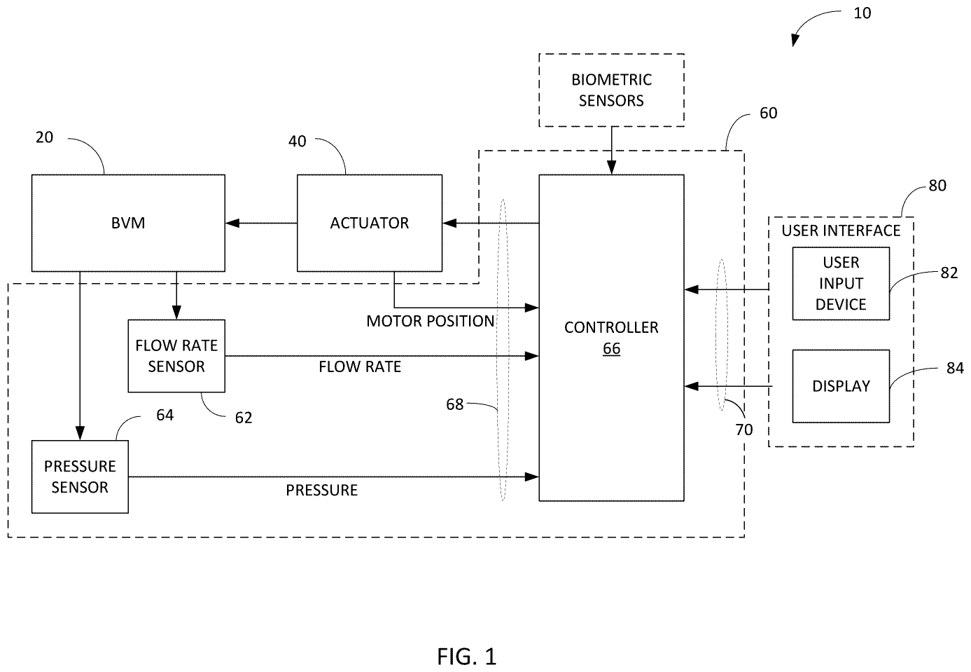

FIG. 1 is a high level block diagram illustrating a patient ventilation system indicated generally by the numeral 10. The patient ventilation system 10 comprises a BVM 20, a mechanical actuator 40, a control system 60, and user interface (UI) 80. The BVM 20 can be operated manually or automatically to provide positive pressure ventilation to a patient that is not breathing or that needs supplemental oxygen. The mechanical actuator 40 provides a mechanism for automating the compression and expansion of the BVM 20 as will be hereinafter described. The control system 60 serves two main functions: to give feedback to the user via the user interface 80 in a manual operating mode and to automate operation of the BVM 20 in an autonomous mode.

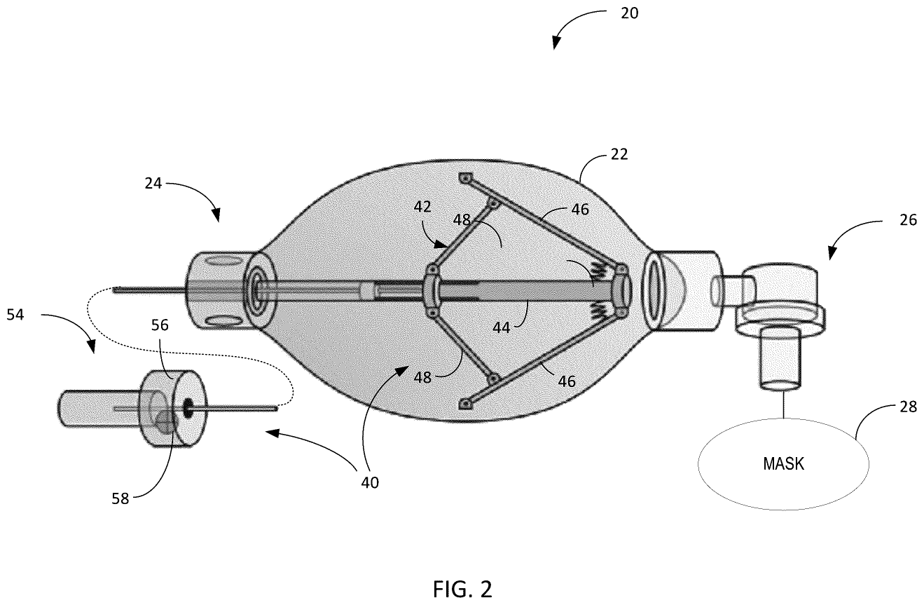

FIG. 2 schematically illustrates the BVM 20 and actuator 40. The BVM 20 comprises a flexible self-inflating bag 22, an air intake valve 24, a patient valve 26 and face mask 28. The flexible self-inflating bag 22 is made of a plastic material that re-expands after being manually collapsed. The self-inflating bag 22 may come in various sizes, e.g., 240 mL for infants, 500 mL for children, and 1600 mL for adults. The air intake valve 24 is disposed at one end of the self-inflating bag 22 and the patient valve 25 is disposed at the opposite end. The air intake valve 24 allows air or oxygen to enter the bag when the bag re-expands after being collapsed. The air intake valve 24 typically includes an oxygen inlet port (not shown) for connection to an oxygen source. The patient valve 26 is a one-way valve that directs the flow of air or oxygen from the self-inflating bag 22 to the face mask 28 when the flexible self-inflating bag 22 is collapsed while preventing exhaled gases from re-entering the flexible self-inflating bag 22. The patient valve 26 includes an exhalation port, pressure limiting valve and adapter for connecting to the face mask 28. The face mask 28 is designed to conform to the patient's face and provide a seal to prevent gases from escaping. During exhalation, gases are directed to the patient valve 26 and exit through the exhalation port in the patient valve 26.

During use, the self-inflating bag 22 is manually or automatically collapsed to force air through the patient valve 26 and into the face mask 28 to provide positive pressure ventilation. When the pressure on the self-inflating bag 22 is released, the self-inflating bag 22 re-expands to draw air in through the air intake 24. Gases exhaled by the patient exit through the exhalation port in the patient valve 26.

Automation of the BVM 20 can be achieved by having a mechanism, referred to herein as an actuator 40, to compress the self-inflating bag 22 of the BVM 20. The actuator 40 can be independent of the self-inflating bag 22, like an external air pump connected to the self-inflating bag 22, or a mechanism that integrates with the self-inflating bag 22. The actuator 40 can be driven by a servo motor and powered by an onboard rechargeable battery (not shown).

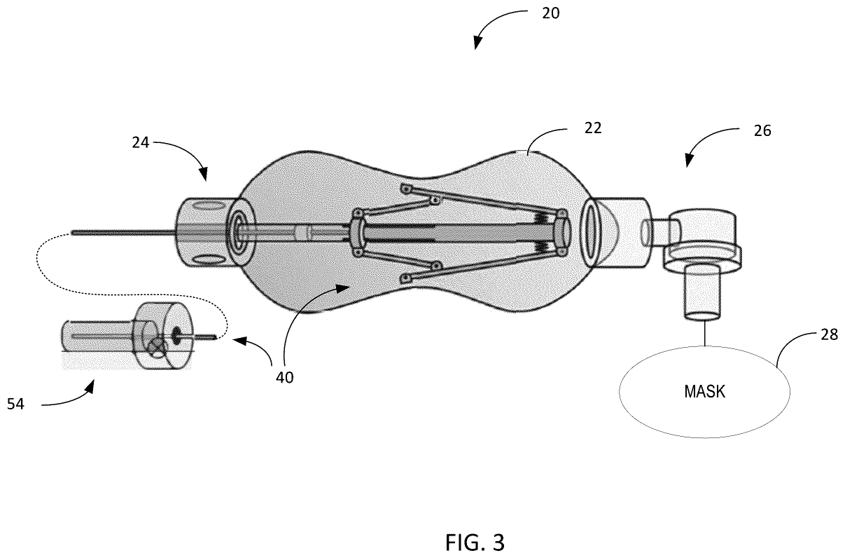

FIGS. 2 and 3 illustrate an exemplary actuator 40 for automating BVM operation. The actuator 40 comprises two main assemblies, an internal link mechanism 42 and a drive unit. The link mechanism 42 comprises a tube 44 that is inserted into the self-inflating bag 22 through the air intake valve 24, two primary linkages 46, two connecting linkages 48, and a slide 50. The two primary linkages 46 are pivotally attached at one end to the tube 42 and at the opposite end to the interior of the self-inflating bag 22 on diametrically opposed sides. The connecting linkages 48 are pivotally attached at one end to the slide 50 and at the opposite end to the primary linkages 46. The slide 50 comprises a collar that slides axially along the length of the tube 44. The slide 50 is connected by a drive rod 52 to the drive unit 54. The drive unit 54 comprises a servomotor 56 and pinion gear 58 that reciprocates the drive rod 52 back and forth in the axial direction.

When the autonomous mode activated, the servomotor 56 turns in a first direction during an inhalation phase and reverses direction during an exhalation phase. In the inhalation phase, the servomotor 56 pulls the internal linkages 46, 48 inward, collapsing the self-inflating bag 22 and forcing air out through the face mask 28. In the exhalation phase, the servomotor 56 reverses direction allowing the self-inflating bag 22 to re-inflate. This process can be repeated, allowing for automated ventilation.

The actuator 40 fits within the dimensions of a typical BVM 20, enabling complex automation and sensor feedback within a form factor that is both portable and familiar to users.

The control system 60 provides feedback to the user via the user interface 80 and automates operation of the BVM 20 in the autonomous mode. The control system 60 comprises a flow rate sensor 62 and a pressure sensor 64 providing feedback to a controller 66, which generates control signals based on the feedback from the sensors 62, 64 to control the servomotor 58. The servomotor 56 and sensors 62, 64 can be coupled to the controller 66 via a wired or wireless interface (e.g., BLUETOOTH) 68. In some embodiments, the control system 60 may further include one or more gas concentration sensors located in the patient valve 26 to measure, for example, the oxygen concentration in the air provided to the patient and the carbon dioxide concentration in the exhaled air.

The flow rate sensor 62 and pressure sensor 64 are disposed in the air flow path between the self-inflating bag 22 and the face mask. For example, the flow rate sensor 46 and pressure sensor 64 can be disposed in the patient valve 26. As air flow through the patient valve 26 from the self-inflating bag into the face mask 28 during the inhalation phase, the flow rate sensor 62 and pressure sensor 64 measure the flow rate and pressure respectively. Exhaled air passes through the same flow rate sensor 62 and pressure sensor 64 in the exhalation phase. The measurements made by the flow rate sensor 62 and pressure sensor 64 are input to the controller 66 and used to make adjustments to the drive signals for the actuator 40.

The user interface 80 provides means to receive user input and to output information to the user for viewing. The user interface 80 can be coupled to the controller 66 via a wired or wireless interface (e.g., BLUETOOTH) 70. The user interface 80 comprises one or more input devices 82 to set and/or adjust the operating parameters of the controller 60, and a display 84 to display information for viewing by the user. The user interface could also provide audible or tactile feedback to the emergency responder in place of or in addition to visual feedback. The user interface 80 can be coupled to the controller 66 by a wired or wireless interface.

The user input devices 82 may comprise one or more push buttons, a keypad, pointing device (e.g., mouse or trackball), touch screen, voice control, or combination thereof. Display 84 may comprise an electronic display such as a light emitting diode (LED) display, liquid crystal display (LCD), or other common type of display. In some embodiments, the display may comprise a touch screen display that also serves as a user input device 82. The main purpose of the display 84 is to display operating parameters or metrics (e.g., airway pressure, tidal volume delivered, and gas concentrations) to provide responders with performance feedback, allowing them to correct inadequate ventilation.

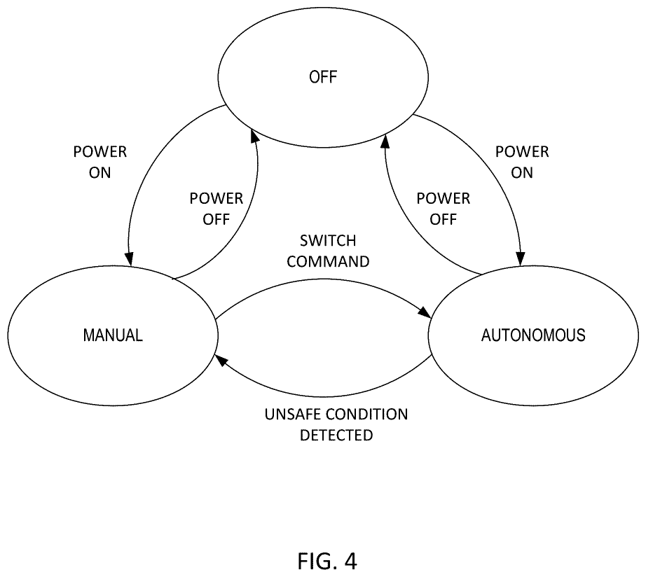

FIG. 4 illustrates the operating states, also referred to as operating modes, of the patient ventilation system 10. The operating modes include a power off state, a manual operating state, and an autonomous state. When the patient ventilation system is powered on, the control system 60 enters either the manual operating mode or autonomous operating mode based on user selection of the operating mode.

In the manual operating mode, the user manually operates the BVM 22 and the sensors 62, 64 provide feedback to the controller 66 to generate performance metrics that can be displayed on the display 84. Based on the feedback, the user can make adjustments to achieve the desired ventilation for the patient. In one embodiment, the displayed information comprises the ventilation rate and tidal volume. The concentrations of oxygen in the inhaled air and the CO2 in the exhaled air could also be displayed. The control system 60 also performs one or more safety checks as hereinafter described and alerts the user if an unsafe condition is detected to prevent injury to the patient.

In the automated mode, the user may enter target ventilation parameters (e.g., ventilation rate and tidal volume) via the user interface 80 and the controller 66 generates drive signals to operate the BVM 20 based on the input parameters. Generally, the controller 66 determines the angular displacement of the servomotor 56 needed to achieve the target tidal volume. Once the target tidal volume is determined, the controller 66 determines the motor speed from the target ventilation rate and angular displacement. The controller then computes the drive signals based on the computed motor speed and angular displacement.

During the autonomous operating mode, the control system 60 monitors the flow rate and pressure from the sensors 62 and 64 and computes the actual ventilation rate and tidal volume from the measured flow rate and timing of the inhalation/exhalation phases of the ventilation cycle. The controller 66 compares the computed values of the ventilation rate and tidal volume to the target values input by the user and computes error values based on the comparison. The control system 60 adjusts the drive signals sent to the actuator 40 based on the error values. For example, if the ventilation rate is low, drive signals are generated to increase the motor speed of the servomotor 56 and shorten the duration of the ventilation cycle. If the ventilation rate is high, drive signals are generated to decrease the speed of the servomotor 56 and increase the duration of the ventilation cycle. As another example, if the tidal volume is low, the control system 60 may adjust the angular displacement of the motor to increase the stroke length of the link mechanism 42, i.e., to increase the amount by which the self-inflating bag 22 is compressed. If the tidal volume is high, the control system 60 may adjust the angular displacement of the motor to decrease the stroke length of the link mechanism 42, i.e., to decrease the amount by which the self-inflating bag 22 is compressed. Note that changing the stroke length of the link mechanism may also require adjustment of the motor speed if the ventilation rate is unchanged.

Automated ventilation fulfills the role of one responder (squeezing the bag at an adequate respiratory rate), allowing a single provider to focus on ensuring leak-proof mask seal around the patient's nose and mouth. This, in combination with sensor feedback to ensure adequate ventilation, minimizes the physical and mental strain on responders and improves patient outcomes.

As shown in FIG. 4, the patient ventilation system may transition directly from the manual operating mode to the autonomous operating mode. In one embodiment, the user input devices 82 include a readily accessible button that is manually pressed by the user to switch from the manual operating mode to the autonomous operating mode. While the BVM 20 is manually operated, the ventilation parameters are displayed to the user on the display 84 so that the user can adjust their performance based on the feedback. During manual operation, the ventilation parameters are saved in memory and continuously updated. Once the user is satisfied with their performance, the user can enable automated ventilation with the press of a button to switch to the autonomous mode. When the user switches to the automated mode, the saved ventilation parameters are taken as the target ventilation parameters. As described above, the controller 66 computes the initial angular displacement and motor speed based on the target ventilation parameters to mimic the user's technique. In effect, the controller 66 “remembers” the user's technique and mirrors the performance of the user (squeeze rate, volume delivered) without the need for the user to manually enter the input parameters.

The patient ventilation system 10 may transition directly from the autonomous operating mode to the manual operating mode. As noted above, the controller 66 performs a series of safety checks in the autonomous mode to avoid injury to the patient. If the controller 66 detects an unsafe condition, the controller 66 alerts the user and switches automatically to the manual operating mode.

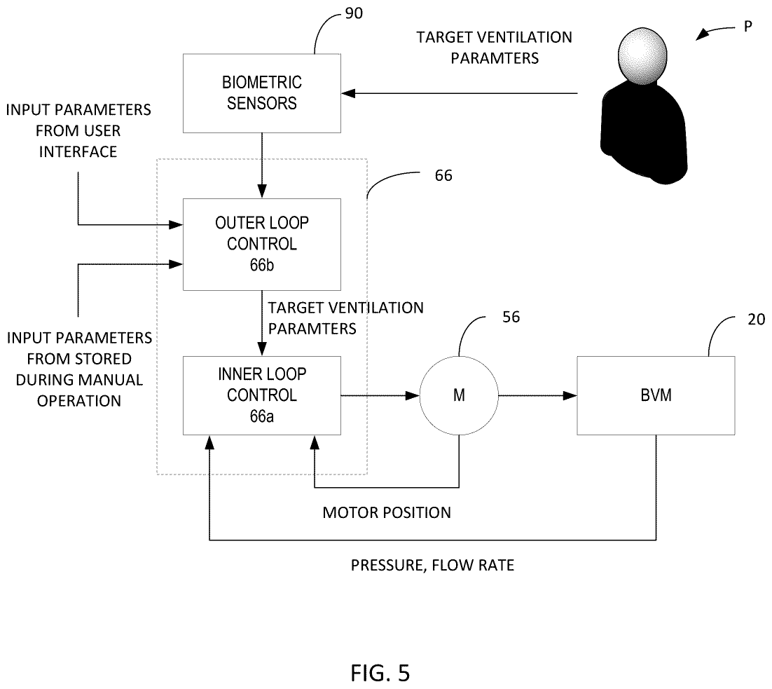

FIG. 5 schematically illustrates a method of controlling the BVM 22. The controller 66 implements an inner loop control mechanism 66a based on feedback from the BVM 20 and an outer loop control mechanism 66b based on input from biometric sensors monitoring the patient P. The biometric sensors 90 may, for example, monitor the patient's pulse rate, oxygen saturation levels, breath rate, etc. The inner loop control mechanism 66a generates drive signals for the motor 56 to operate the BVM 20 based on target ventilation parameters provided by the outer loop control 66b and feedback from the BVM 20. As previously noted, the measured flow rate provided by the flow rate sensor 62 is used to calculate an estimated ventilation rate and tidal volume. The estimated ventilation rate and tidal volume are compared to the target values and the drive signals are adjusted based on the comparison. The outer loop control 66b receives input parameters from the user via the user interface 80 or reads the input parameters from memory in the case where the user switches from manual mode to automatic mode. The input parameters are used as the initial target ventilation rate and tidal volume. During autonomous operation, biometric sensors 90 provide feedback indicative of the patient's condition. The feedback from the biometric sensors 90 is used to adjust the target ventilation rate and target tidal volume. These new target values will then be used by the inner loop control 66a to control the motor 56.

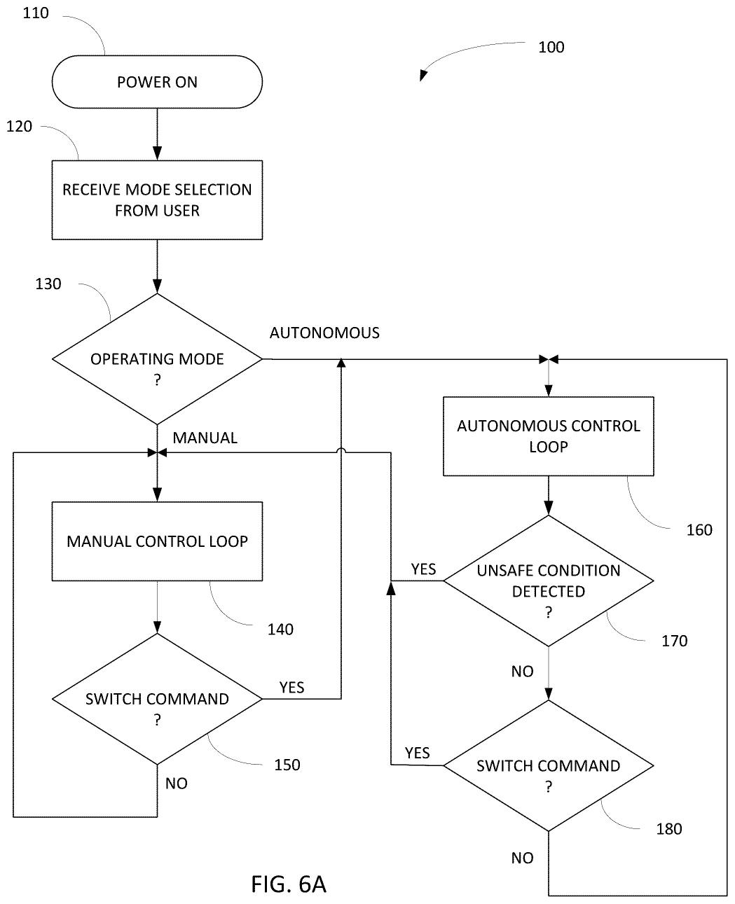

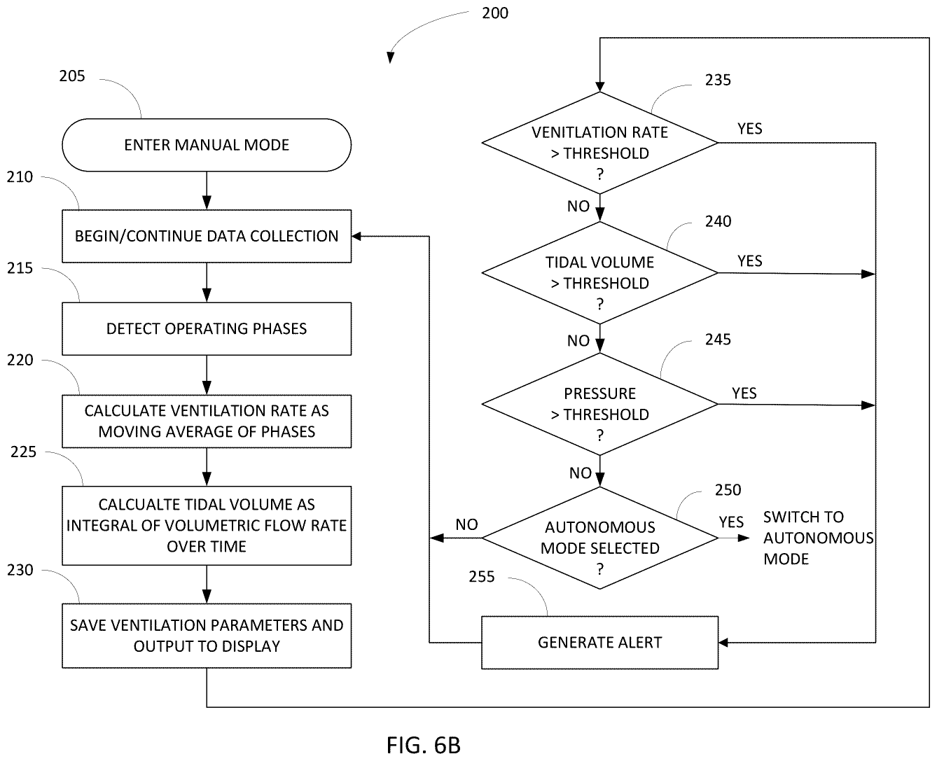

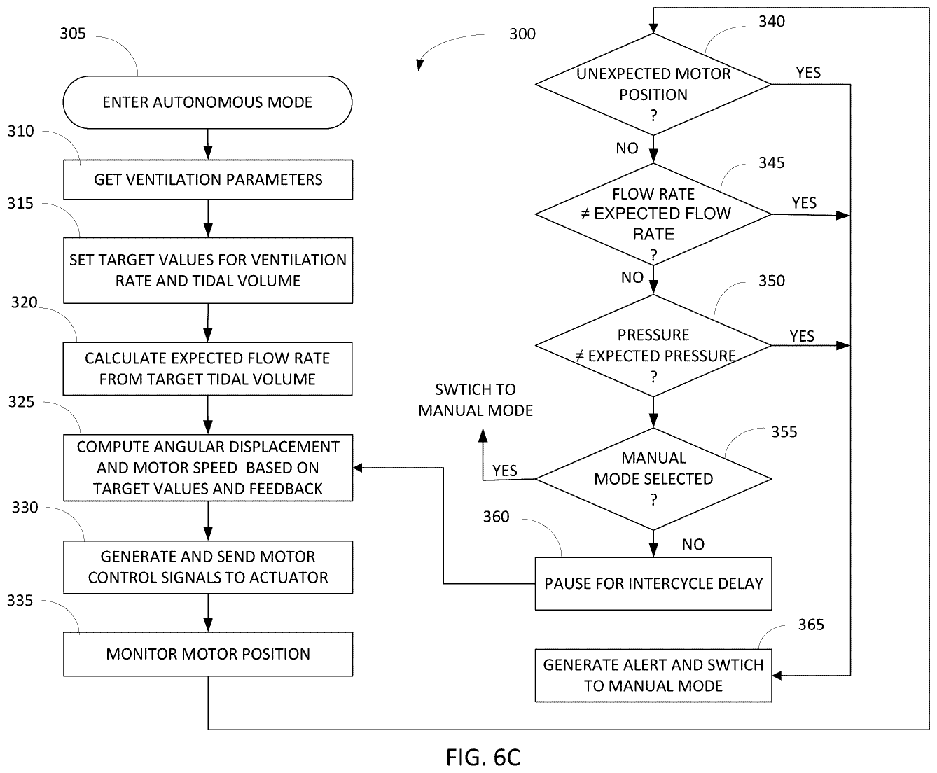

FIGS. 6A-6C illustrate an exemplary control procedure 100 implemented by the controller 66. FIG. 6A illustrates the overall procedure at a high level. FIG. 4B illustrates a manual control loop 200 for the manual mode and FIG. 4C illustrates an autonomous control loop 300 for autonomous mode.

Referring to FIG. 6A, the procedure 100 begins when the device is powered on (block 110). It is presumed that the face mask 28 has been affixed to the patient. The user selects the desired operating mode via the user interface 80 (block 110). The controller 66 determines the operating mode based on the user's selection (block 130). If the manual operating mode is selected, the control logic enters the manual control loop 200 (block 140). If the autonomous operating mode is selected the control logic enters the autonomous control loop 300 (block 160). While in the manual control loop 200, the controller 66 checks for switch command from the user instructing the controller 66 to switch from manual control to autonomous control, e.g., by pressing a button (block 150). If a switch command is detected, the controller 66 enters the autonomous control loop 300 (block 160). Controller 66 stores the ventilation parameters detected during manual ventilation of the patient. When it switches to autonomous mode, controller 66 retrieves the stored ventilation parameters and mirrors the performance of the user. While in the autonomous operating mode, the controller 66 checks for an unsafe condition (block 170). If an unsafe condition is detected, the controller 66 automatically switches to the manual operating mode. Also, a user may switch back to manual operating mode. Controller 66 checks for a command to switch back to manual mode (block 180). If a command to switch is detected, controller switches back to manual mode. Otherwise, controller 66 remains in autonomous mode.

FIG. 6B illustrates one example of the control logic for the manual operating mode. The manual operating mode may be initially selected by the user, or may be triggered by detection of an unsafe condition while in the autonomous operating mode. In either case, the controller 66 enters the manual operating mode and begins collecting data from the flow rate sensor and pressure sensor (blocks 205, 210). The controller 66 analyzes the collected data to detect the inhalation and exhalation phases of operation (block 215). The controller computes moving averages of the phases, which is taken as the ventilation rate, i.e., number of ventilation cycles per minute (block 220). The controller 66 estimates the tidal volume as the integral of the flow rate over time (block 225). The computed ventilation parameters are saved in memory and output for display to the user along with other data, such as pressure, gas concentrations, etc. (block 230).

After the ventilation parameters are computed, the controller 66 performs a series of checks in order to ensure patient safety. In the first check, the controller 66 compares the computed ventilation rate to a threshold (block 235). Separate thresholds can be used to define a range. For example, an upper threshold and a lower threshold can be defined for the ventilation rate. If the moving average is outside the range defined by the thresholds, the controller alerts the user, e.g., by generating an alarm (block 255). In the second check, the controller 66 compares the tidal volume to a threshold and alerts the user if the threshold is exceeded (blocks 240, 255). In the third check, the controller 66 compares the measured pressure to a threshold and alerts the user if the threshold is exceeded (blocks 245, 255). After these safety checks are performed, the controller 66 determines whether the user has commanded a switch to the autonomous operating mode, for example, by pressing a button (block 250). If not the control loop 200 repeats until the autonomous mode is enabled or until the system is powered off. If the user switches to the autonomous mode, the manual control loop 200 terminates and control passes to the autonomous control loop 300.

FIG. 6C illustrates one example of the control logic for the autonomous operating mode. The autonomous operating mode may be initially entered from the power off state or from the manual operating mode (block 305). Upon entry into the autonomous operating mode, the controller gets the initial ventilation parameters (block 310). When switching from the manual operating mode, the saved ventilation parameters stored in manual operating mode (if switching) may be taken as the initial ventilation parameters so there is no need for the user to enter the ventilation parameters. Otherwise, the controller 66 prompts the user to enter the initial ventilation parameters. In some embodiments, the user may override the stored ventilation parameters by manually entering new ventilation parameters. The controller 66 sets the target ventilation rate and target tidal volume based on the input ventilation parameters and calculates the expected flow rate (block 315). The expected flow rate can be calculated from the input ventilation rate and tidal volume. More particularly, the duration of the ventilation cycle phases can be computed from the ventilation rate and multiplied by the tidal volume to get an estimate of the expected flow rate. The controller 66 estimates the angular displacement and motor speed for the servomotor 56 to achieve the desired ventilation rate and tidal volume (block 325). As noted, the angular displacement can be estimated from the tidal volume and a reference table relating angular displacement to tidal volumes. Once the angular displacement is determined, the motor speed is computed based on the angular displacement and ventilation rate. The controller then generates and sends the drive signals to the servomotor 56 (block 330). While the servomotor 56 reciprocates the linkage mechanism in the actuator 40, it provides feedback to the controller 66 on the motor position (block 335).

While in the autonomous operating mode, the controller 66 also performs a series of safety checks to prevent injury to the patient. First, the controller 66 checks the motor position to make sure that it is within expected bounds (block 340). Second, the controller 66 checks the flow rate to make sure that it does not deviate from the expected flow rate by more than a predetermined threshold (block 345). In other embodiments, controller 66 may check whether the flow rate exceeds a predetermined threshold that might injure the patient. Third, the controller 66 checks the pressure to make sure that the pressure does not deviate from the expected pressure by more than a threshold (block 350). In other embodiments, controller 66 may check whether the pressure exceeds a predetermined threshold that might injure the patient. If any safety checks fails, the controller generates an alert and switches to the manual operating mode (block 365). If the performance is as expected, the controller 66 checks for a command to switch to a manual mode (block 355). If a command to switch is detected, controller 66 returns to autonomous mode. Otherwise, controller 66 pauses for an intercycle delay (block 360) and control returns to block 325.

Following each cycle, the controller 66 receives the flow rate and pressure from the sensors 62 and 64 and computes the actual ventilation rate and tidal volume from the measured flow rate and timing of the inhalation/exhalation phases of the ventilation cycle. The controller 66 compares the computed values of the ventilation rate and tidal volume to the target values input by the user and adjusts the drive signals sent to the actuator 40 based on the error values.

The patient ventilation system 10 allows a single emergency responder to operate the BVM 22 to provide respiratory aid to a patient. Displaying performance metrics enables the emergency responder to adjust their technique to provide adequate air flow to the patient while reducing the chance of patient injury and the emotional strain on the emergency responder. Once the user is satisfied with their performance, they can then enable automated ventilation to provide consistent ventilation to the patient. The controller recognizes the user's technique and with the press of a button, the control system of the DIVA mirrors the performance of the user. The autonomous mode fulfills the role of one responder (squeezing the bag at an adequate respiratory rate) allowing a single provider to focus operate the BVM 20 so that other responder can focus on other tasks.

What is claimed is:

1. A method of controlling a bag valve mask having a manual operating mode and an autonomous operating mode, the method comprising:
receive sensor data from one or more sensors;
in the manual operating mode, determining one or more ventilation parameters based on the sensor data while the bag valve mask is being manually operated by a user;
storing values of the one ventilation parameters measured during the manual operation of the bag valve mask;
receiving a control signal to switch from the manual operating mode to the autonomous mode;
switching to the autonomous operating mode responsive to the control signal,
upon switching to the autonomous operating mode, setting an initial target value for the one or more ventilation parameter based on the stored values of the ventilation parameters; and
generating control parameters for an actuator based on the initial target values of the one or more ventilation parameters.

2. The method of claim 1, wherein one of the ventilation parameters comprises a ventilation rate.

3. The method of claim 2, further comprising:
detecting phases of a ventilation cycle from the sensor data; and
determining the ventilation rate based on the phases of the ventilation cycle.

4. The method of claim 3, determining the ventilation rate based on the phases of the ventilation cycle comprises calculating the ventilation rate as a moving average of the phases of the ventilation cycle.

5. The method of claim 1, wherein one of the stored ventilation parameters comprises a tidal volume.

6. The method of claim 1, wherein the ventilation parameters comprise a ventilation rate and a tidal volume.

7. The method of claim 1, further comprising, in the manual operating mode:
continuously monitoring patient ventilation based on the sensor data.

8. The method of claim 1, further comprising displaying the one or more ventilation parameters on a display.

9. The method of claim 1, further comprising alerting the user when one of the ventilation parameters is outside a desired operating range.

10. The method of claim 1, further comprising:
receiving biometric data indicative of a patient's condition from one or more biometric sensors; and
perform outer loop control by adjusting the target ventilation parameters based on the biometric data.

11. The method of claim 10, further comprising:
continuously collecting sensor data from a flow rate sensor and pressure sensor for each or one more ventilation cycles; and
in an autonomous operating mode, perform inner loop control by adjusting the control parameters to the actuator based on current target values for the ventilation parameters and the sensor data from the flow rate sensor.

12. The method of claim 1, wherein the actuator includes a motor and further comprising:
monitoring motor position during the autonomous operating mode; and
alerting the user when the motor position is outside a desired operating range.

13. The method of claim 12, further comprising:
monitoring a flow rate during the autonomous operating mode; and
alerting the user when the flow rate is outside a desired operating range.

14. The method of claim 12, further comprising:
monitoring an air pressure during the autonomous operating mode; and
alerting the user when the air pressure exceeds a pressure threshold.

15. A control system for a bag valve mask having a manual operating mode and an autonomous operating mode, the control system comprising:
one or more sensors for collecting sensor data indicative of operating conditions of the bag valve mask; and
a controller configured to:
receive the sensor data from the one or more sensors;
in the manual operating mode, determine one or more ventilation parameters based on the sensor data while the bag valve mask is being manually operated by a user;
store values of the ventilation parameters measured during the manual operation of the bag valve mask;
receive a control signal to switch from the manual operating mode to the autonomous mode;
switch from the manual operating mode to the autonomous operating mode responsive to the control signal,
upon switching to the autonomous operating mode, set an initial target value for a control parameter based on the stored values of the ventilation parameters; and
generate control parameters for an actuator based on the initial target values of the one or more ventilation parameters.

16. The control system of claim 15, wherein the controller is further configured to:
detect phases of a ventilation cycle from the sensor data; and
determine the ventilation rate based on the phases of the ventilation cycle.

17. The control system of claim 15, wherein the controller is configured to determine the ventilation rate based on the phases of the ventilation cycle comprises calculating the ventilation rate as a moving average of the phases of the ventilation cycle.

18. The control system of claim 15, wherein one of the stored ventilation parameters comprises a tidal volume.

19. The control system of claim 15, wherein the ventilation parameters comprise a ventilation rate and a tidal volume.

20. The control system of claim 15, wherein the controller is further configured to continuously monitor patient ventilation based on the sensor data in the manual operating mode.

21. The control system of claim 15, wherein the controller is further configured to display the one or more ventilation parameters on a display.

22. The control system of claim 15, wherein the controller is further configured to alert the user when one of the ventilation parameters is outside a desired operating range in the autonomous operating mode.

23. The control system of claim 15, wherein the controller is further configured to:
receive biometric data indicative of a patient's condition from one or more biometric sensors; and
perform outer loop control to adjust the target ventilation parameters based on the biometric data.

24. The control system of claim 15, further comprising:
continuously collecting sensor data from a flow rate sensor and pressure sensor for each or one more ventilation cycles; and
in an autonomous operating mode, perform inner loop control to adjust the control parameters to the actuator based on current target values for the ventilation parameters and the sensor data from the flow rate sensor.

25. The control system of claim 15, wherein the actuator includes a motor and further comprising:
monitoring motor position during the autonomous operating mode; and
alerting the user when the motor position is outside a desired operating range.

26. The control system of claim 15, further comprising:
monitoring a flow rate during the autonomous operating mode; and
alerting the user when the flow rate is outside a desired operating range.

27. The control system of claim 25, further comprising:
monitoring an air pressure during the autonomous operating mode; and
alerting the user when the air pressure exceeds a pressure threshold.
